(19) United States
(12) Patent Application Publication
(10) Pub. No.: US 2020/0102283 A1
(43) Pub. Date: Apr. 2, 2020

(54) Methods for Obtaining Purified Cannabis Extracts and THCA Crystals

(71) Applicants: Clare J. DIBBLE, Arvada, CO (US); Isaac B. COLE, Berkeley, CO (US)

(72) Inventors: Clare J. DIBBLE, Arvada, CO (US); Isaac B. COLE, Berkeley, CO (US)

(21) Appl. No.: 16/686,080

(22) Filed: Nov. 15, 2019

Related U.S. Application Data
(63) Continuation of application No. 15/202,385, filed on Jul. 5, 2016, now abandoned.

(51) Int. Cl. C07D 311/80; B01D 11/02; B01D 21/00; B01D 9/00

(52) U.S. Cl. CPC C07D 311/80; B01D 11/0288; B01D 9/0004; B01D 21/0012; B01D 9/004; B01D 21/009

(57) ABSTRACT

The present invention includes a method for obtaining a higher purity cannabinoid solvent extract from a plant which comprises cannabinoids and/or terpenes. A solvent extraction is performed on the optionally dried plant material, followed by a step of removing high molecular weight impurities by a cooling step. Following the cooling step, the precipitate is removed and a higher quality filtrate is obtained which contains higher levels of purity of cannabinoids and/or terpenes than the starting solvent extract. The methods of the invention also include a method for obtaining crystallized THCa, which comprises obtaining a filtrate by the methods disclosed herein, or obtaining a solvent extract, and allowing crystallization of the THCa to occur. The filtrate, crystallized THCa, and residual filtrate remaining after crystallization of THCa can be used as starting materials for products that include cannabinoids and/or terpenes.

### CROSS-REFERENCE TO RELATED APPLICATIONS

This application is a continuation of U.S. application Ser. No. 15/202,385, filed Jul. 5, 2016, which claims the benefit of U.S. Provisional Application Ser. No. 62/188,965, filed Jul. 6, 2015. Both of these applications are hereby incorporated by reference in their entireties.

### BACKGROUND

Cannabinoids are a diverse class of chemical compounds that act as a ligand to the brain's cannabinoid receptors. The clinical usefulness of the cannabinoids, including Δ⁹-tetrahydrocannabinol (Δ⁹-THC), to provide analgesia, help alleviate nausea and emesis, as well as stimulate appetite has been well-recognized. Cannabinoids offer a variety of pharmacological benefits, including, but not limited to, anti-spasmodic, anti-inflammatory, anti-convulsant, anti-psychotic, anti-oxidant, neuroprotective, anti-inflammatory, anti-cancer, and immunomodulatory effects.

The cannabis plant is the primary source of cannabinoids. Like all terrestrial plants, lignocellulose gives the plant its structure. Lignocellulosic material is composed of carbohydrate polymers such as cellulose and hemicellulose, and an aromatic polymer called lignin. Lignin is a constituent of the cell walls of almost all dry land plant cell walls. It is the second most abundant natural polymer in the world, surpassed only by cellulose. The non-structural chemical components of plant cells are much more highly variable from plant to plant and within different parts of a plant. These are referred to as extractives, referencing the relative ease with which they can be separated from the lignocellulose that composes the structure of the plant. Cannabinoids are the class of chemicals that make the cannabis plant unique, but terpenoids, sugars, fatty acids, flavonoids, other hydrocarbons, nitrogenous compounds, and amino acids have also been identified in cannabis plants.

The principle cannabinoids present in herbal cannabis are cannabinoid acids Δ⁹-tetrahydrocannabinolic acid (Δ⁹-THCa) and cannabidiolic acid (CBDa) with small amounts of the respective neutral (decarboxylated) cannabinoids. In addition, cannabis may contain lower levels of other minor cannabinoids.

In general, a crude extract of cannabis can be made via solvent extraction. The resultant oil, or cannabis resin, is a dark brown, viscous and sticky oil and generally contains up to about 75% of THC (or THCa), depending on the extraction conditions. The balance of the cannabis resin generally contains other cannabinoids, terpenoids and a significant amount of other materials that originated in the plant that are not known to have therapeutic value. In particular, the extract may contain lignin, lignans, gums, pigments, and lecithin. Lignin, as a structural polymer, would not typically be extracted with polar solvents such as water, but may be extracted with non-polar solvents used to extract resins.

Crude extracts from cannabis plants are often used by patients suffering from diseases and disorders, such crude products are less suitable for use in pharmaceutical formulations. It would be preferable to have purified forms of certain cannabinoids. Fractional distillation, immiscible liquid-liquid separation, or preparative and flash chromatography have been employed individually or in combination to separate desirable components of plant extracts from less desirable counterparts in other pharmaceutical plant preparations and natural products like essential oils. However, these techniques either tend to be difficult to scale and make continuous, or tend to degrade the molecules of interest.

Therefore, improved methods for removing plant material such as lignin, lignans, gums and lecithins from cannabis oil, and improved methods for obtaining purified THC or THCa, are desired in the art.

The present invention is directed toward overcoming one or more of the problems discussed above.

### SUMMARY OF THE EMBODIMENTS

In one embodiment, the present invention discloses a method for obtaining a higher purity cannabinoid solvent extract from a plant which comprises at least one cannabinoid. This method includes the steps of performing a solvent extraction of the plant to yield a solvent extract; a step of cooling the solvent extract; and a step of removing the precipitate from the cooled solvent extract to yield a solvent extract filtrate, wherein the solvent extract filtrate has a higher purity of the at least one cannabinoid. The initial precipitate includes substances that are capable of carbonizing rather than completely evaporating when heated, such as lignocellulosic material, lignin, lignans, and/or lecithin. The solvent may include a short chain hydrocarbon, such as, for example, butane; carbon dioxide, an alcohol, or a terpene. The step of cooling the solvent extract involves cooling until the solute forms a solid but the temperature and pressure are in a range where the solvent remains fluid. For the example of butane solvents, this may include cooling the solvent extract to a temperature of between about −50° C. and about −85° C. for a time period of between about 30 minutes to about 6 hours. The plant may be cannabis or hemp, and the cannabinoid may be tetrahydrocannabinolic acid (THCa). This method may further optionally include the step of crystallizing the THCa from the solvent extract filtrate. Alternatively, the method includes crystallizing the THCa directly from the solvent extract, particularly where the plant (or plant parts) comprise a high percentage of cannabinoids and/or THCa. For example, to obtain crystals of THCa, the solvent extract filtrate may be cooled to a temperature of about −75° C. for a time period of between about 12 hours and three days to obtain crystals of THCa of greater than about 95% purity. Optionally, THCa may be precipitated directly from the extract making the filtrate lower in (THC+THCa) but replete with cannabinoids and terpenes from the plant.

Various modifications and additions can be made to the embodiments discussed without departing from the scope of the invention. For example, while the embodiments described above refer to particular features, the scope of this invention also included embodiments having different combination of features and embodiments that do not include all of the above described features.

### DETAILED DESCRIPTION

Disclosed herein are methods for improving the purification of plant extracts via removal of undesirable impurities and, in the case of cannabis extracts, subsequent selective isolation of tetrahydrocannabinolic acid (THCa) from other cannabinoids and terpenes. The improved process for purifying plant extracts can be conducted in open or closed systems and in a batch or continuous manner. The extract to be purified can be from any vegetation, but this method is particularly suited to cannabis.

While all natural product precursors exhibit inherent variation, it is desirable to obtain a consistent product from any part of the plant that contains that product within a single harvest and from different harvests. While a variety of extraction techniques have become commonplace in cannabis, a secondary separation step is rarely employed. To create high quality products, manufacturers favor extractions from bud and higher grade trim and with targeted solvents like lighter hydrocarbons (propane instead of butane) to help minimize the removal of these impurities that contribute to off flavors and processing inconsistencies. Surprisingly, it is possible to remove the undesirable extractives by sedimentation and removal of said impurities promotes the crystallization of THCa, when it is present. The present inventor also found methods to crystallize THCa from, for example, extracted bud and higher-quality trim.

In one embodiment, the present invention includes a method for obtaining a higher purity cannabinoid solvent extract from a plant which comprises at least one cannabinoid. The method includes the steps of performing a solvent extraction of the plant to yield a solvent extract, cooling the solvent extract; and removing the precipitate from the cooled solvent extract to yield a solvent extract filtrate. Optionally, the cannabis supernatant can be cooled another time to yield a precipitate of high purity THCa crystals and a residual filtrate enriched in other cannabinoids and terpenes. The methods of the invention result in obtaining a solvent extract filtrate which has a higher purity of the at least one cannabinoid. These steps are discussed hereinbelow.

The solvent extract may comprise tetrahydrocannabinol, cannabidiol, and the carboxylic acids thereof from cannabis plant material.

Exemplary cannabinoids useful for the present invention include cannabinols. In one embodiment, the invention includes tetrahydrocannabinols, including the most commonly known cannabinoid, tetrahydrocannabinol (THC). The most potent stereoisomer occurs naturally as Δ⁹-THC where the two chiral centers at C-6a and C-10a are in the trans configuration as the (−)-trans-isomer, and this stereoisomer is also known as dronobinol. There are seven double bond isomers in the partially saturated carbocylic ring including Δ^{6a,7}-tetrahydrocannabinol, Δ⁷-tetrahydrocannabinol, Δ⁸-tetrahydrocannabinol, Δ^{9,11}-tetrahydrocannabinol, Δ¹⁰-tetrahydrocannabinol, Δ¹⁰-tetrahydrocannabinol, and Δ^{6a,10a}-tetrahydrocannabinol, using the dibenzopyran numbering:

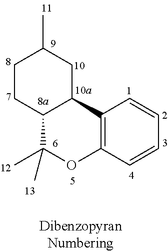 [SMILES: CC1CC[C@@H]2[C@@H](C1)c1ccccc1OC2(C)C]

The cannabinols have the following general structure:

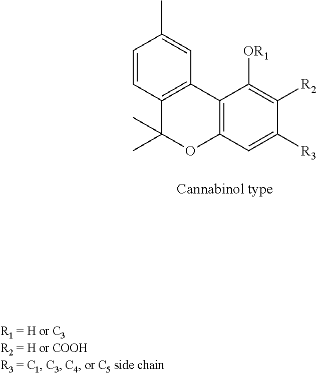

Below is Δ⁹-tetrahydrocannabinol.

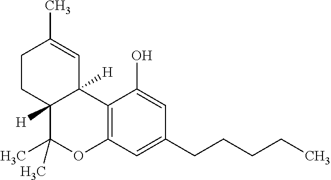 [SMILES: CCCCCc1cc(O)c2c(c1)OC(C)(C)[C@@H]1CCC(C)=C[C@@H]21]

Tetrahydrocannabinol, such as Δ⁹ THC, helps reduce nausea and vomiting, which is particularly helpful to patients undergoing chemotherapy for cancer. Patients suffering from AIDS often experience a lack of appetite, of which tetrahydrocannabinol is also helpful in counteracting. Tetrahydrocannabinol is also useful for glaucoma relief.

THC may be derived from Cannabis sativa or Cannabis indica, for example.

The cannabinoids include cannabinoids which have a carboxylic acid substituent, also known as cannabinoid acids, such as tetrahydrocannabinolic acid (THCa) which has a carboxylic acid at R². These carboxylic acids are designated as “a”. For example, CBD occurs as CBDa in the cannabis plant. The 2-carboxylic acids of the cannabinoids can be decarboxylated by heat, light, or alkaline conditions to their respective decarboxylated compounds, such as to Δ⁹-THC. See below for the structure of Δ⁹-THCa.

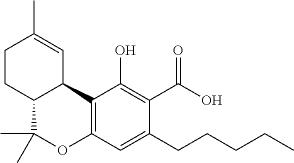 [SMILES: CCCCCc1cc2c(c(O)c1C(=O)O)[C@@H]1C=C(C)CC[C@H]1C(C)(C)O2]

Decarboxylation of the cannabinoid acids to the corresponding phenols occurs over time, upon heating, or under alkaline conditions. Heating for 5 minutes at a temperature of 200-210 ° C. will accomplish decarboxylation. THCa is the non-activated, non-psychotropic acid form of THC. THCa is a known anti-inflammatory and provides many of the same benefits of THC but without psychotropic side effects. THCa not only has anti-proliferative abilities that are crucial in helping inhibit the growth of cancerous cells, but also, it has anti-spasmodic abilities that helps subdue muscle spasms and therefore has potential use among epileptic patients.

Cannabinoids may also occur as their pharmaceutically acceptable salts. As used herein, the expression “tetrahydrocannabinol” or “THC”—where not otherwise specified—is to encompass any isomers thereof, in particular double bond isomers.

A cannabinol useful for the present invention also includes tetrahydrocannabivarin (THCv) having a propyl side chain.

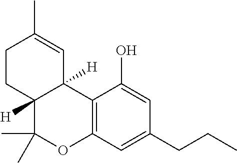 [SMILES: CCCc1cc(O)c2c(c1)OC(C)(C)[C@@H]1CCC(C)=C[C@@H]21]

Tetrahydrocannabivarin—THCV is structurally similar to THC, but acts an antagonist to the CB1&CB2 receptors in the body. Given this, recent studies have shown that THCV is an excellent appetite suppressant as it blocks the rewarding sensations experienced when eating. THCV also holds anti-convulsive properties useful for treating epilepsy. While psychoactive, THCV lends itself to a shorter, psychedelic, clear-headed effect which is shorter lasting that THC.

A cannabinoid useful for the present invention also includes cannabinol (CBN).

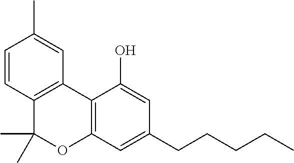 [SMILES: CCCCCc1cc(O)c2c(c1)OC(C)(C)c1ccc(C)cc1-2]

CBN's primary effects are as an anti-epileptic, anti-spasmodic and reliever of intra-ocular pressure. Recent studies suggest that CBN can be administered as an antidepressant, can be used to prevent convulsions and to sedate patients experiencing pain. It is ideal for those suffering from glaucoma, inflammation, and insomnia.

A cannabinoid useful for the present invention also includes a cannabidiol type.

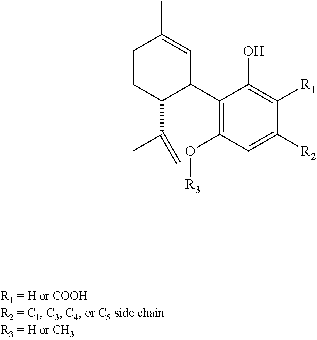

A cannabinoid useful for the present invention also includes the naturally occurring cannabidiol type also called (−)-trans-cannabidiol (CBD).

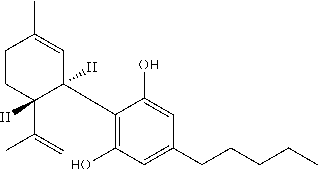 [SMILES: C=C(C)[C@@H]1CCC(C)=C[C@H]1c1c(O)cc(CCCCC)cc1O]

CBD can occur in up to 40% of the cannabinoid extracts from cannabis. CBD generally occurs in the cannabis plant prior to processing as CBDa which has a carboxylic acid at R¹. The 2-carboxylic acids of the cannabinoids can be decarboxylated by heat, light, or alkaline conditions to their respective decarboxylated compounds.

CBD and CBDa have been shown effective in treating inflammation, diabetes, cancer, mood disorders (PTSD to ADD) and neurodegenerative diseases such as Alzheimer's. It has been shown to have anti-convulsive, anti-anxiety, anti-psychotic, anti-nausea and anti-rheumatoid arthritic and sedative properties, and a clinical trial showed that it eliminates anxiety and other unpleasant psychological side effects. CBD does not display the psychoactive effects of Δ⁹-THC. CBD was found in one study to be more effective than aspirin for pain relief and reducing inflammation. CBD has been shown to be a potent antioxidant as well as having neuroprotective and anti-inflammatory uses.

A cannabinoid useful for the present invention also includes cannabichromene type, or

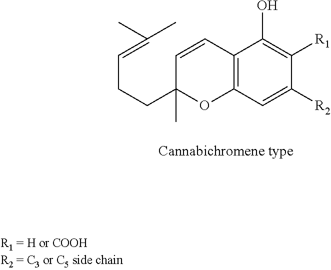

An exemplary cannabichromene (CBC) is shown below:

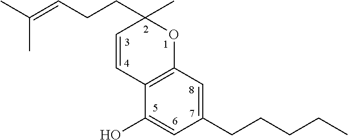 [SMILES: CCCCCc1cc(O)c2c(c1)OC(C)(CCC=C(C)C)C=C2]

CBC, like THC and CBD, results from CBCa. CBC has been shown to inhibit the growth of cancerous tumors due to its interaction with anadamide, a human endocannabinoid. It is also an inflammation and pain inhibitor and has been successful for treating migraines and stimulating bone growth. Due to its small quantity in the cannabis plant, CBC works best in conjunction with CBD and THC.

The plant which comprises at least one cannabinoid optionally further comprises at least one terpene and/or terpenoid. The methods of the present invention are also optionally useful to obtain a higher purity of terpene(s). Terpenes are a diverse group of organic hydrocarbons derived from 5-carbon isoprene units and are produced by a wide variety of plants. Terpenes are naturally present in cannabis; however, they can be removed during the extraction process.

In one embodiment, the terpene/terpenoid includes limonene. Limonene is a colorless liquid hydrocarbon classified as a cyclic terpene. The more common D-isomer possesses a strong smell of oranges and a bitter taste. Limonene is a chiral molecule. Biological sources produce one enantiomer—the principal industrial source—citrus fruit, contains D-limonene ((+)-limonene), which is the (R)-enantiomer (CAS number 5989-27-5, EINECS number 227-813-5). Racemic limonene is known as dipentene. Its IUPAC name is 1-methyl-4-(1-methylethenyl)-cyclohexene. It is also known as 4-isopropenyl-1-methylcyclohexenep-Menth-1,8-dieneRacemic: DL-limonene; dipentene.

In another embodiment, the terpene/terpenoid includes linalool. It is also known as β-linalool, linalyl alcohol, linaloyl oxide, p-linalool, allo-ocimenol, and 3,7-dimethyl-1,6-octadien-3-ol. Its IUPAC name is 3,7-dimethylocta-1,6-dien-3-ol.

In another embodiment, the terpene/terpenoid includes myrcene. Myrcene, or β-myrcene. α-Myrcene is the name for the structural isomer 2-methyl-6-methylene-1,7-octadiene, which is not found in nature and is little used. Its IUPAC name is 7-methyl-3-methylene-1,6-octadiene.

In another embodiment, the terpene/terpenoid includes α-Pinene. Pinene is found in conifer, pine and orange. α-Pinene is a major constituent in turpentine. Its IUPAC name is (1S,5S)-2,6,6-Trimethylbicyclo[3.1.1]hept-2-ene ((−)-α-Pinene).

In another embodiment, the terpene/terpenoid includes β-Pinene. Its IUPAC name is 6,6-dimethyl-2-methylenebicyclo[3.1.1]heptane and is also known as 2(10)-Pinene; Nopinene; Pseudopinene. It is found in cumin, lemon, pine and other plants.

In another embodiment, the terpene/terpenoid includes caryophyllene, also known as β-caryophyllene. Caryophyllene is a natural bicyclic sesquiterpene that is a constituent of many essential oils, including clove, cannabis, rosemary and hops. It is usually found as a mixture with isocaryophyllene (the cis double bond isomer) and α-humulene, a ring-opened isomer. Caryophyllene is notable for having a rare cyclobutane ring. Its IUPAC name is 4,11,11-trimethyl-8-methylene-bicyclo[7.2.0]undec-4-ene.

Caryophyllene is known to be one of the compounds that contribute to the spiciness of black pepper. In another embodiment, the terpene/terpenoid includes citral. Citral, or 3,7-dimethyl-2,6-octadienal or lemonal, is either a pair, or a mixture of terpenoids with the molecular formula C₁₀H₁₆O. The two compounds are double bond isomers. The E-isomer is known as geranial or citral A. The Z-isomer is known as neral or citral B. Its IUPAC name is 3,7-dimethylocta-2,6-dienal. It is also known as citral, geranial, neral, geranialdehyde.

In another embodiment, the terpene/terpenoid includes humulene. Humulene, also known as α-humulene or α-caryophyllene, is a naturally occurring monocyclic sesquiterpene (C₁₅H₂₄), which is an 11-membered ring consisting of 3 isoprene units containing three nonconjugated C═C double bonds, two of them being triply substituted and one being doubly substituted. It was first found in the essential oils of Humulus lupulus (hops). Humulene is an isomer of β-caryophyllene, and the two are often found together as a mixture in many aromatic plants.

Other exemplary terpenes/terpenoids include menthol, eucalyptol, borneol, pulegone, sabinene, terpineol and thymol.

The methods of the present invention may be used with a plant which comprises at least one cannabinoid. A plant that comprises at least one cannabinoid includes Cannabis (hemp). For the botanical and chemotaxonomical differentiation of the genus Cannabis there are two different concepts. One differentiates between three species, Cannabis sativa Linnaeus, Cannabis indica LAM, and Cannabis ruderalis, while a different theory only sees the existence of the one collective species Cannabis sativa L. made up of the subspecies Cannabis sativa ssp. sativa and ssp. indica. Moreover the cannabis plant is differentiated into a drug type and a fiber type, with differentiation being performed on the basis of the quantity ratio of the main cannabinoids, cannabidiol (CBD) and Δ⁹-tetrahydrocannabinol (Δ⁹-THC). Fiber hemp, whose cultivation is permitted for fiber production, must not exceed a Δ⁹-THC content of 0.3% relative to the dry plant mass, while the drug type may exhibit a Δ⁹-THC content of approx. 5%-15% relative to the dry plant mass.

The ratio of Δ⁹-THC to CBD in fiber hemp is mostly less than 1.5. The varieties rich in Δ⁹-THC may reach a ratio of 2:1 to 7:1. Cannabis sativa L. occurs worldwide in all warm and moderate zones with the exception of the humid tropical rain forests. It is an annual to biennial, anemogamous herb which may attain a height of up to 8 m. The dioecous, rarely monecious inflorescences contain the active cannabinoids in the resin which is mainly secreted by the numerous glandular bracts in the leaf axils. As a general rule, all the plant parts of Cannabis sativa L. with the exception of the seeds may contain cannabinoids. The highest cannabinoid concentrations are found in the floral bracts and fruit stalks. The leaves have a low content of cannabinoids as a function of leaf age, while the stalk and particularly the root exhibit clearly lower cannabinoid contents.

In one embodiment, the present invention includes a step of a solvent extraction of the plant which comprises cannabinoids. The term “plant” includes a plant or plant part such as bark, wood, leaves, stems, roots, flowers, fruits, seeds, berries or parts thereof).

Where the starting plant material is freshly harvested or wet, the plant material may be subjected to a drying step to remove excess moisture or a freezing step to immobilize moisture in the plant. Therefore, in one embodiment, the cannabis is dried. In another embodiment, the cannabis is frozen. Optionally, the plant material comprises dried bud, trim, or fan leaves, which are optionally milled.

The plant material is optionally has not been subject to a decarboxylation step and the cannabinoids are primarily present as their carboxylic acid forms. In other embodiments, the plant material has been subject to a decarboxylation step and the cannabinoids are present as their neutral forms. If the extract has been subject to a decarboxylation step or is extracted from hemp or other plant material that does not contain THCa, only the first sedimentation step is employed, as THCa will not crystalize if it is not present in the filtrate. In the case that relatively pure THCa is desired, the material may be retained in its acid form by processing fresh or recently dried materials, not exposing the material or extracts to heat or UV light, and/or maintaining any inert atmosphere that reducing the probability of oxidation reactions, as is known in the art.

In one embodiment, the method includes a step of performing a solvent extraction of the plant to obtain a solvent extract. Generally, the solvent extraction step can be carried out by methods that are known in the art. Extraction solvents for use in the methods of the present invention include non-polar solvents such as short chain hydrocarbons (including, for example, propane, butane, hexane, and the like), alcohols such as ethanol or methanol, and liquid and/or supercritical carbon dioxide, steam, and terpenes.

Generally, to perform the solvent extraction step, the solvent is passed over hand harvested or milled plant materials in order to extract concentrated fractions. Bulk solids can be retained by a mesh screen, or any other known methods for filtration or separation between liquids and solids may be used.

As one example of the process, dried cannabis material (bud, trim, or fan leaves), which is optionally milled (bowl trim, and/ or blended in a blender), is packed into an extraction column, for example, about 50 g plant matter is packed into a 1.5 inch diameter aluminum column 12 inches in length. Alternatively, 80 to 200 g biomass containing cannabis is placed into a 2 inch diameter stainless steel column between 12 and 30 inches in length. The extraction column packed with the biomass can be, for example, supported by a stand with a screen secured on the bottom and rubber stopper with a center hole containing a nozzle on the top. 600 mL of cooled 99+% pure n-butane or (or 95% n-butane with 4.1+% iso-butane) (for example, 10 ° C. or colder) is allowed to pass over the column. The liquid that flows from the packed bed can be collected in a beaker below the screen end of the tube. The process can be repeated several times and the total liquid from the multiple runs can be combined. In one embodiment, 200 g or more of extracted plant matter is processed and 25 g or more product (THCa) is obtained.

Table 1 below shows the fate of initial and extracted solids and THC as a function of dry weight in a typical butane extraction contrasted with the products of the methods disclosed herein (Table 2). The initial sediment (precipitate after the first precipitation step) includes the molecules that contribute to the dark color of certain extracts and those that carbonize and leave residual solids during vaporization of a sample. While some cannabinoids are removed with this sediment, their concentration is lower than the concentration in the original extract, leaving a filtrate enriched in at least one cannabinoid. In the case where no THCa is present in the initial extract, such as hemp or a plant other than a species of cannabis, the resulting filtrate is not subjected to a secondary separation step. If THCa is present in the initial filtrate, chilling the initial filtrate a second time after removing the initial sediment results in precipitated THCa and a residual filtrate relatively lower in THC and replete with any other cannabinoids or terpenes present in the original plant extract.

TABLE 1
Yields according to prior art processes

| Material | Total Solids (g) | % (THC + THCa) | (THC + THCa) (g) |
|---|---|---|---|
| Starting trim | 100 | 10% | 10 |
| Typical butane | 12 | 75% | 9 |
| extract | | | |

TABLE 2
Yields according to the instant invention

| Material | Total Solids (g) | % (THC + THCa) | (THC + THCa) (g) |
|---|---|---|---|
| Starting trim | 100 | 10% | 10 |
| Typical butane | 12 | 75% | 9 |
| extract | | | |
| Initial sediment | 1.5 | 60% | 0.9 |
| Initial filtrate | 10.5 | 77% | 8.1 |
| THCa | 6.5 | 95% | 6.2 |
| precipitate | | | |
| Residual | 4 | 48% | 1.9 |
| filtrate | | | |
| Total | 12 | 75% | 9 |

Typically in cannabis extracts, such a carrier solvent is typically removed immediately following an extraction process. In the present process, the cannabis solvent extract can be used as collected in the solvent extraction step without removing the solvent. The ratio of solvent to dry weight of plant matter extract can be adjusted by adding more of the same solvent, a different solvent, or removing some proportion of solvent. Alternatively, the solvent may be removed from the extract and the extract re-solubilized in a different solvent.

The solvent extract, either used as collected, or adjusted in volume or type of solvent as discussed above, is then used in the additional step(s) of the method.

In this step of the method, the solvent extract is treated to remove higher molecular weight impurities that create carbonized residuals when the rest of the sample is vaporized. Without being bound by theory, the present inventor believes that the high molecular weight impurities to be lignin, lecithin, and/or other undesirable, high molecular weight materials that were extracted by the solvent. These higher weight impurities can comprise, for example, such materials common to plants such as lignin, lignans, pigments, gums, lignocellulosic material, and lecithin. Lignin is commonly understood as a complex polymer of aromatic alcohols and is a component of the cell walls of plants. Plant lignans are polyphenolic substances derived from phenylalanine via dimerization of substituted cinnamic alcohols, known as monolignols. Plant pigments include chlorophyl and other carotenoids that absorb light to catalyze photosynthesis. Gums include complex polysaccharides. Lignocellulosic material is composed of carbohydrate polymers such as cellulose and hemicellulose, crosslinked to an aromatic polymer (lignin). The solvent extract will also contain lower molecular weight components such as cannabinoids and volatile terpenes.

In one embodiment, the solvent extract is cooled to allow for precipitation of the higher molecular weight impurities. In the cooling step, the temperature of the solvent extract or co-solvents should be maintained in such a way that the mixture is chilled but the solvent remains fluid, allowing impurities to condense and settle to the bottom of the container.

In one example of the present invention, a container (such as a beaker) containing a butane solvent extract is allowed to sit directly on dry ice in a cooler for 1-4 hours. The cooler is optionally between about −40° C. and −70° C. The temperature can be varied during the process, and is optionally carried out at an average temperature of less than about 10° C., less than about 0° C., less than about −10° C., less than −20° C., less than about −30° C., less than about −40° C., less than about −50° C., less than about −60° C., less than about −70° C., or less than about −80° C. In another embodiment, the temperature at which the cooling takes place is between about −50° C. and about −85° C. The cooling step may take place for between about 1 minute and 24 hours, between about 10 minutes and about 18 hours, between about 30 minutes and about 12 hours, between about one hour and about 8 hours, between about 2 hours and about four hours. Alternatively, the cooling step may take place for longer than 10 minutes, longer than 30 minutes, longer than about an hour, longer than about two hours, longer than about three hours, longer than about four hours, longer than about six hours, longer than about eight hours, longer than about 12 hours, longer than about 18 hours, or about twenty four hours or longer. As an example of a device to facilitate cooling the solvent extract on dry ice, Pelican ProGear Elite Marine Deluxe Coolers work especially well for maintaining low temperatures when filled with dry ice. Alternatively methods for cooling the solvent extract may also be used, such as storing in a cold environment such as in a refrigerator or freezer, or by use of liquid nitrogen.

The present invention also includes removing a precipitate from the cooled solvent extract. In some embodiments, the high molecular weight impurities present in the solvent extract turn dark when exposed to air. The precipitate can be removed from the cooled solvent extract by any methods known in the art. For example, the precipitate can be removed by filtration, or by transferring the supernatant to a clean vessel. This process is not intended for removal of entrained solids, but can handle small amounts of material that may inadvertently be included in the mixture. In one embodiment of the present invention, the impurities portion constitutes 1-15% of the total extract weight and can be as much as 60% THCa. To discard the impurities represents a loss of 10% of the total extracted THCa or less.

As an example of the removal step, after precipitate has formed (on the bottom of the beaker in this example), the solvent extract is filtered through a vacuum assisted Buchner funnel using 12.5 cm diameter 101 fast filter paper and coffee filter and, if possible, taking care not to disturb the cake on the bottom of the beaker. In some embodiments, the sediment forms bubbles during filtration, indicating solvent evaporation and possibly a surfactant nature to the sediment.

Optionally, the cooling process can be repeated as many times as necessary for maximum removal of the initial precipitate. Optionally, sedimentation (precipitation) can be repeated until the filtrate is optically clear. In this embodiment, the filtrate is returned to a clean beaker and the cooling step is repeated, followed by the step of removing the precipitate. For example, the beaker can be cooled another 1-3 hours and filtered again in a Buchner funnel with a coffee filter and slow quantitative filter. The present inventor has found that after two filterings, the solvent and extract are typically significantly more pure. The more optically clear the solvent is, the better the separation has gone. If vaporized, this filtrate leaves no residue or a light waxy white residue, but no carbonized black residuals.

The solvent extract filtrate typically contains a higher percentage of cannabinoid(s) and/or terpenes than the initial solvent extract. In the example illustrated in Table 2, the filtrate THCa concentration has increased from 75% to 77%. The solvent extract filtrate, following this step, may be optionally dried by methods known in the art to remove the solvent. The dried solvent extract filtrate can then be used as desired, as a typical oil, shatter or wax that has not undergone a separation process. For example, the butane can be evaporated from filtrate and the extract gently heated in a desiccator or vacuum oven to convert any THCa to THC. Optionally hemp products or products from other plant extracts would terminate this process here.

The methods of the present invention optionally further comprise crystallization (precipitation) of THCa. In this step, the solvent extract filtrate can be treated to allow the THCa to crystallize out of solution. The solvent extract filtrate can be collected and used in the crystallization step without any further modification. Alternatively, the solvent extract filtrate can have the ratio of solvent to dry weight of filtrate adjusted by adding more of the same solvent, a different solvent (co-solvent), or removing some proportion of solvent. Alternatively, the solvent may be removed from the filtrate and the filtrate re-solubilized in a different solvent or solvents for the crystallization step. Crystallization is preferably carried out in highly non-polar solvents, such as hydrocarbons such as butane, oils such as vegetable oils and coconut oil or terpenes.

Thus, the solvent extract filtrate, either used as collected, or adjusted in volume or type of solvent as discussed above, is then optionally used in the crystallization step.

The crystallization step is enhanced after the strong tasting, dark brown material (without being bound by theory, understood as high molecular weight lignin, lignans, gums, lignocellulosic material, and the like) has been removed, as described hereinabove, resulting in higher purity THCa. The crystallization step can be performed by methods as known in the art.

In one embodiment, the solvent extract filtrate is cooled to allow for crystallization of the THCa. In the cooling step, the temperature of the solvent extract filtrate should be maintained in such a way that the mixture is chilled but the solvent remains fluid, allowing THCa to crystallize and settle to the bottom of the container.

In one example of the crystallization step, a container (such as a beaker) containing the solvent extract filtrate is allowed to sit in a cooler containing dry ice for between about 12 hours and several days. The cooler is optionally between about −40° C. and −70° C. In one embodiment, the temperature is about −75 ° C. The temperature can be varied during the process, and is optionally carried out at an average temperature of less than about 10° C., less than about 0° C., less than about −10° C., less than −20° C., less than about −30° C., less than about −40° C., less than about −50° C., less than about −60° C., less than about −70° C., or less than about −80° C. In another embodiment, the temperature at which the cooling takes place is between about −50° C. and about −85° C. The cooling step may take place for between about one hour and one week, between about 10 hours and about four days, between about one day and about three days. Alternatively, the cooling step may take place for longer than one hour longer than about ten hours, longer than about 18 hours, longer than about 24 hours, longer than about 36 hours, longer than about 48 hours, longer than about 72 hours, longer than about 96 hours, longer than about 120 hours, or about 168 hours or longer. Alternatively methods for cooling the solvent extract filtrate may also be used, such as storing in a cold environment such as in a refrigerator or freezer, or by use of liquid nitrogen. Optionally, the crystallization step is performed without vibrating or disturbing the solvent extract filtrate. The preferred crystallization container material is glass. Optionally, the crystallization may be performed under pressure or vacuum.

In one embodiment, the crystallization step is encouraged and/or enhanced by increasing surface area. Methods to increase surface area for crystallization are known in the art, such as glass beads, which are optionally added prior to the crystallization.

Crystals of THCa can be harvested by methods known in the art. In one example, crystals may be obtained by filtering solvent and extract and capturing the retentate as well as removing crystals by scraping them from the glass beads through a sieve with a metal spatula. Optionally, the crystallization step may be repeated as many times as desired. The mother liquor can be subjected to another cooling step to test if crystals will continue to form. The present inventor has found that if yellow oil is present with the crystals, the separation from the other terpenes has ceased to be effective. Crystallization should optionally be terminated before or when extracted oils begin to condense and foul the pure THCa.

The present inventor has found two morphologies of crystals, “sheet” and “ball” crystals. Which morphology dominates seems to be influenced by the process conditions and the quality of the starting material.

In one embodiment, after the crystals of THCa have been collected according to the methods of the present invention, the remaining extract can be collected and used by evaporating the solvent. The residual filtrate was found to contain cannabinoids and terpenes extracted from the original bud or trim, and THC and THCa that did not crystallize during the course of the run. The residual filtrate can also be incorporated into finished products of their own, but retain some of the characteristics of the original material instead of being quality independent from the source materials, like the crystallized THCa.

The products made by the processes of the instant invention, e.g., crystallized THCa, solvent extract filtrate, or residual filtrate, for example, may be used in the acid form, or converted to the neutral forms by methods known in the art. The products made by the processes of the instant invention may be incorporated into any product or formulation, such as, for example, those products or formulations that are typically known to incorporate a cannabinoid. Convenient formulations include tablets, capsules, oils, gels, lozenges, troches, hard candies, nutritional bars, nutritional drinks, metered sprays, creams, suppositories, transdermal patches, among others. The compositions may be combined with a pharmaceutically acceptable excipient such as gelatin, oil(s), and/or other pharmaceutically active agent(s). The crystallized THCa may be used in the acid form, or converted to the neutral form by methods known in the art.

The products may be advantageously combined and/or used in combination with other therapeutic or prophylactic agents, such as one or more cannabinoids and/or terpenes. In many instances, administration in conjunction with the subject products enhances the efficacy of such agents.

The inventor has found that following the steps of the invention, 95+% pure THCa is readily crystallized from solution in quantities greater than 50% of the total extracted THCa. The balance of the THC and THCa remains in the solvent with the rest of the plant extract in the residual filtrate, which is enriched in cannabinoids and terpenes relative to the original plant extract. The residual filtrate, following crystallization of THCa, is relatively depleted in THCa, but will contain other cannabinoids and terpenes. The residual filtrate may be combined with other materials and/or formed into products or formulations as described herein.

The present invention provides for the purity of the crystallized THCa to be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% pure (w/w)

Optionally, any product should have solvent thoroughly removed.

Accordingly, the present invention includes a method for obtaining crystallized THCa from a plant which comprises at least one cannabinoid. These steps include performing a solvent extraction of the plant to yield a solvent extract, cooling the solvent extract, removing the precipitate from the cooled solvent extract to yield a solvent extract filtrate; allowing THCa to crystallize from the solvent extract filtrate; and collecting the crystallized THCa.

The present invention also includes a method for obtaining crystallized THCa from a plant which comprises at least one cannabinoid, such as THCa, directly from the solvent extract. In this method, a plant or specific plant parts, such as bud and/or trim, that are relatively enriched for one or more cannabinoids, such as THCa, are optionally used. This method encompasses directly crystallizing THCa from the solvent extract, by performing a solvent extraction of the plant or specific plant parts in accordance with the methods of the invention. The method then comprises cooling the solvent extract to allow the THCa to crystallize from the solvent extract according to the methods disclosed in the present invention, and collecting the crystallized THCa. The invention also encompasses THCa obtained by the methods of the invention.

All percentages and amounts in the present application, if not otherwise defined, are to be defined as weight percents (w/w).

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

### EXAMPLES

The following examples are provided for illustrative purposes only and are not intended to limit the scope of the invention.

### Example 1

Extraction. Dried cannabis material (bud, trim, or fan leaves, milled) was obtained. 50 g plant matter was added to a 1.5 inch diameter aluminum column 12 inches in length, supported by a stand with a screen secured on the bottom and rubber stopper with a center hole containing a nozzle on the top. In a well ventilated area, two 300 mL cans of 10° C. 99+% pure n-butane were poured into the top of the column, about 5-10 minutes. The extract was collected in a beaker and placed on dry ice in a cooler. The extraction was repeated two times and the extracts were combined prior to the separation step.

Separation. The beaker containing the extract was allowed to sit directly on dry ice in a Pelican ProGear Elite Marine Deluxe Coolers cooler for 4 hours at approximately −70° C. Precipitate was observed on the bottom of the beaker. The extract was filtered through a vacuum assisted Buchner funnel using 12.5 cm diameter 101 fast filter paper and coffee filter taking care not to disturb the cake on the bottom of the beaker. The filtrate was returned to a clean beaker and put back in the cooler on top of the dry ice for 3 hours and filtered again in a Buchner funnel with a coffee filter and slow quantitative filter. After two filterings, the solvent extracts were optically clear. The retentate turned brown upon the solvent evaporation, is believed to be lignin, lecithin, and/or other undesirable, high molecular weight materials that were extracted by the solvent.

Crystallization. THCa, the acid precursor to THC, was crystallized out of solution using the filtered solvent extract. Glass beads were added to the beaker before putting it into an undisturbed deep freeze (−75° C.). Crystals formed in between 12 hours and several days. The crystals were harvested by filtering solvent and extract and capturing the retentate as well as removing crystals by scraping them from the glass beads through a sieve with a metal spatula. After collecting the crystals, the butane and extract mixture were returned to the deep freeze to collect additional crystals. Two morphologies of crystals were observed, “sheet” and “ball” crystals. The THCa crystals were 98+% pure THCa in quantities greater than 50% of the total extracted THCa. The balance of the THC and THCa remained in the solvent with the rest of the plant extract residual filtrate which is enriched in cannabinoids and terpenes relative to the original plant extract.

Termination. When the crystallization ceased to provide a clean separation, the remaining extract was collected by evaporating the solvent. This residual filtrate contained cannabinoids and terpenes extracted from the original bud or trim, and THC and THCa that did not crystallize during the course of the run.

The below table shows yields from each step of the process.

| Material | Total Solids (g) | % (THC + THCa) | (THC + THCa) (g) |
|---|---|---|---|
| Starting trim | 100 | 10% | 10 |
| Typical butane | 12 | 75% | 9 |
| extract | | | |
| Initial sediment | 1.5 | 60% | 0.9 |
| Initial filtrate | 10.5 | 77% | 8.1 |
| THCa | 6.5 | 95% | 6.2 |
| precipitate | | | |
| Residual | 4 | 48% | 1.9 |
| filtrate | | | |
| Total | 12 | 75% | 9 |

The description of the various embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art.

The embodiments described and shown in the figures were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

1. A method for obtaining crystallized acid (THCa) from a plant which comprises at least one cannabinoid, consisting the steps of:
a) performing a solvent extraction of the plant, wherein the plant is selected from leaves, stems, trim, or fan leaves, to yield a solvent extract wherein the solvent is butane;
b) cooling the solvent extract to a temperature between approximately −50° C. to −85° C. for a time period of about 30 minutes to about 6 hours;
c) obtaining a precipitate comprising lignocellulosic materials and removing the precipitate from the cooled solvent extract to yield a solvent extract filtrate;
d) cooling the solvent extract filtrate to a temperature of about −75° C. for between about 10 hours and about four days allowing THCa to crystallize from the solvent extract filtrate; and
e) collecting the crystallized THCa, wherein the crystallized THCa has an enhanced purity of greater than 95%.

2. The method of claim 14, wherein the THCa is selectively crystallized away from other soluble cannabinoids.

3. The method of claim 14, wherein the method of collecting the crystallized THCa comprises separating the crystals of THCa from the solvent extract filtrate.

4. The method of claim 14, wherein the solvent extract is cooled to a temperature of approximately −70° C. for a time period of about 4 hours.
